(19) United States
(12) Patent Application Publication
(10) Pub. No.: US 2007/0281032 A1
(43) Pub. Date: Dec. 6, 2007

(54) Method for generating polymeric wear particles

(76) Inventor: Hsu-Wei Fang, Taipei City (TW)

Correspondence Address: Dykema Gossett, PLLC;Suite 300 West, 1300 I Street, N.W., Washington, DC, 20005-3306, US

(21) Appl. No.: 11/447,173

(22) Filed: Jun. 6, 2006

(51) Int. Cl. A61K 9/28; B05D 3/00

(52) U.S. Cl. 424/489; 427/2.14

(57) ABSTRACT

In a method for generating polymeric wear particles for use in animal experiments as polymeric medical implant pre-clinical testing, microfabrication technique is used to design different mask patterns and obtain a uniformly sized and oriented microfabricated surface, which is used with a reciprocating wear tester to conduct an experiment on wear, so as to generate wear particles having specific size and morphology. A large quantity of wear particles could be generated within a shortened time. Separating and filtering procedures are included to produce wear particles having more uniform size, so as to meet the standards of pre-clinical test and satisfy requirements in different clinical tests. The method includes three major steps, namely, producing a microfabricated surface, conducting an experiment on wear, and collecting generated wear particles.

### FIELD OF THE INVENTION

The present invention relates to a method for generating polymeric wear particles for use in animal experiments as polymeric medical implant pre-clinical testing, and more particularly to a method for generating polymeric wear particles that employs microfabrication technique to design different mask patterns and obtain uniformly sized and oriented microfabricated surfaces, which are then used with a reciprocating wear tester to conduct an experiment on wear, so as to generate a large quantity of wear particles of specific sizes and profiles within a shortened time. Separating and filtering procedures are included to obtain wear particles having more uniform size, so as to meet the standards of pre-clinical tests and satisfy requirements in different clinical tests.

### BACKGROUND OF THE INVENTION

An artificial implant in human body would generate wear particles at two contacting surfaces thereof due to friction in the course of motion. The wear particles present in human body would induce a series of physiological reactions. For instance, the wear particles of ultra-high molecular weight polyethylene (UFMWPE), which is one of the materials for artificial joint, would cause osteolysis and accordingly, loosening and damage of the artificial joint. For this reason, experimental results of biological reactions induced by wear particles must be provided to an authority in charge for examination before a newly developed orthopedics-related implant can be introduced into the market. The relevant research and development institute would conduct experiments on a related simulator, such as an artificial knee joint simulator, to get information on the morphology of the generated wear particles. However, since the quantity of the experimentally generated wear particles is very small and not sufficient for use in an animal experiment as the pre-clinical test, it is very difficult to propel the commercialization of the new implant product.

In a prior research by Shanhbag et al., UHMWPE resin granules are ground at low temperature to obtain relatively small wear particles, which are filtered with a membrane filter to remove particles having relatively large size. However, the yield of particles with desired smaller size is low and the produced particles have varying shapes with particle sizes ranged from 0.1 μm to 0.33 μm and an average size of 0.23 μm. With the above method, it is uneasy to obtain particles of similar shape or controlled size. Therefore, the above method is not suitable for generating wear particles having specific size and shape, and fails to support systematic researches of biological reactions induced by differently sized and shaped wear particles.

It is therefore tried by the inventor to employ the advanced semiconductor processing techniques to produce a uniform-sized and shaped processing surface, which is used with a wear tester to generate differently sized and shaped wear particles under control, so as to satisfy different requirements in clinical tests.

### SUMMARY OF THE INVENTION

A primary object of the present invention is to provide a method for generating polymeric wear particles for use in clinical test, while the generated polymeric wear particles have size and shape that could be precisely controlled. In the method of the present invention, the photolithography in the semiconductor processing technique is employed to produce microfabricated surfaces of different sizes and shapes, and the produced microfabricated surfaces and polymeric materials are caused to abrade each other, so as to generate polymeric wear particles. With the semi-conductor processing technique, patterns having uniform size and orientation may be designed for producing microfabricated surfaces with highly uniform size and specific three-dimensional geometrical shape. When the microfabricated surfaces are used in wear experiments, uniform-sized and shapes wear particles could be quickly obtained. The wear particles are then dispersed and filtered using a supersonic oscillating screener to provide a large quantity of uniform-sized wear particles sufficient for use in the clinical test to meet the standards set by Food and Drug Administration, USA, so that biological tests using the wear particles may be carried out.

The method for generating polymeric wear particles according to the present invention includes three major steps, namely, producing a microfabricated surface, conducting an experiment on wear, and collecting generated wear particles.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is a flowchart showing the major steps included in the method for generating polymeric wear particles for clinical test according to the present invention;

FIG. 2 shows a design of mask pattern on a microfabricated surface for generating polymeric wear particles for clinical test according to the present invention;

FIG. 3 is a top view of a microfabricated surface used in the present invention observed with a scanning electronic microscope;

FIG. 4 is a top view of one cutter formed on the microfabricated surface of FIG. 3;

FIG. 5 is another top view of the cutter of FIG. 4;

FIG. 6 is a schematic top perspective view showing a microfabricated surface with cutters formed thereon for use in the present invention; and

FIG. 7 is a schematic side view of a reciprocating wear tester used in the present invention.

### DETAILED DESCRIPTION OF THE PREFERRED EMBODIMENTS

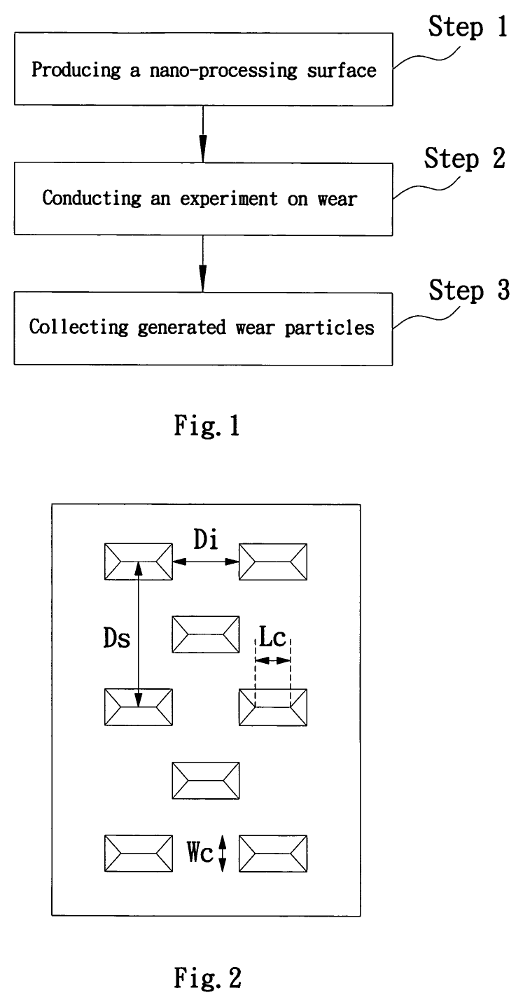

Please refer to FIG. 1 that is a flowchart showing three major steps included in a method for generating polymeric wear particles according to the present invention, namely, producing a microfabricated surface (Step 1), conducting an experiment on wear (Step 2), and collecting generated wear particles (Step 3).

In the Step 1 of producing a microfabricated surface, the following steps are further included:

(11): Preparing a material and designing a mask. A suitable material for a substrate of the microfabricated surface may be selected from silicon wafer, stainless steel, high speed steel, or precision ceramics. The selected substrate is wash cleaned and dipped in a 1:1 mixture of acetone and hexane, subjected to supersonic oscillation for 10 minutes, and then dipped in a detergent and subjected to supersonic oscillation for 10 minutes, and finally washed clean with deionized (DI) water and blown dry using nitrogen. Meanwhile, use the L-edit software to plot a mask pattern having a size and shape determined according to the desired size and shape of the wear particles, and a feature size of 5 μm. Triangular or square mask patterns having different lengths of 60, 20, 10, and 5 μm are separately designed. FIG. 2 shows a design of mask pattern on a microfabricated surface for generating polymeric wear particles for clinical test according to the present invention. Where, Lc is the length of a cutter formed on the microfabricated surface, Wc is the width of the cutter, Ds is the stroke of the cutter, and Di is the interval between two adjacent cutters.

(12): Forming a protective masking layer on the substrate by oxidation-diffusion. A silicon wafer for use as the substrate is sent into a high-temperature reaction furnace, so that a layer of silicon dioxide is formed on the silicon surface of the silicon wafer through reaction of oxygen with silicon under the high temperature. The temperature of the high-temperature furnace is 1050° C. A silicon dioxide layer about 0.5±0.01 μm in thickness is formed in the thermal oxidation process after 60 minutes.

(13): Carrying out a photolithographing process. A two-dimensional (2D) pattern designed in the step (11) is transferred onto the substrate employing the photolithography through the following steps:

(131) Coating photoresist step, which further includes the following steps:
(1311) putting the wafer on a hot plate having a temperature of 120° to bake the wafer for 30 minutes, so as to remove moistures from the surface of the wafer;
(1312) spinning a spin coater at 4000 rpm for 30 seconds to spin coat a layer of hexamethyldisilazane (HMDS) on the silicon wafer, so as to enhance the adsorption of the photoresist to the wafer surface;
(1313) putting the wafer on a vacuum grip device in the spin coater, so that the wafer is sucked to the device under high-speed spinning; using an injector to inject about 3 ml of AZ6112 positive photoresist on a central area of the wafer, and allowing the positive photoresist to spread slowly; carrying out the spin coating in two stages; in the first stage, spinning the spin coater at a low speed of 500 rpm for 10 seconds to coat the photoresist on the wafer; in the second stage, spinning the spin coater at a high speed of 4000 rpm for 30 seconds to control a thickness of the photoresist coating; and
(1314) soft baking the wafer on the hot plate at 90° C. for 5 minutes, so as to remove the solvent in the photoresist while increase the adhesion of the coated photoresist to the wafer and enhance the stability of the photoresist;

(132) Aligning step: turning on a mask aligner when a mercury lamp (I-line) has become stabilized; positioning the mask in the mask aligner, and putting the wafer on a vacuum grip device;

(133) Exposing step: selecting hard contact exposure, so that the light source for exposure is not easily dispersed; and exposing the photoresist coated on the wafer to light for 0.3 second;

(134) Developing step, which further includes the following steps:
(1341) basing on the fact that the molecular bonds of the photoresist at the portions that are not protected by the mask pattern would be broken by the light in the exposing step, and the broken molecular bonds would be dissolved in a developing process while the unexposed portion of photoresist would remain on the wafer surface, dipping the exposed wafer in AZ developer and oscillating for 90 seconds to remove the exposed portions of the positive photoresist, washing the wafer with DI water and blowing dry the wafer, so that the mask pattern is transferred onto the wafer; and
(1342) hard baking the wafer on a hot plate at a temperature higher than 100° C. for 10 minutes, so as to remove any residual solvent and increase the adhesion of the photoresist to the wafer as well as an acid etching resistance of the photoresist;

(14): Carrying out an etching process to produce a three-dimensional picture on the substrate by etching technique, which further includes the following steps:

(141): dipping the wafer having the transferred mask pattern in BOE etching liquid (NH₄F:HF=6:1 by volume) for about 4 minutes and 30 seconds at an etch rate from 1000 to 1200 Å/min, determining the completion of the etching process by observing whether the wafer is unwetted by water, and the silicon dioxide layer unprotected by the photoresist is removed;

(142): Subjecting the wafer to acetone supersonic oscillation for 5 minutes, washing off any residual photoresist, and flushing the wafer surface with DI water;

(143): using an HNA etching liquid (HF:HNO₃:CH₃COOH=8:75:17 by volume) to etch the silicon substrate shielded by the silicon dioxide layer on the wafer for about 2 minutes at an etch rate of 700 Å/min, so as to obtain cutters having desired height and angles when the silicon dioxide layer is etched off;

(144): observing the etching process with an optical microscope, so as to avoid the wafer from being excessively etched; and

(145): vacuum sputtering a chromium layer of 5 nm in thickness on the wafer to give the formed cutters an enhanced wear-resisting property;

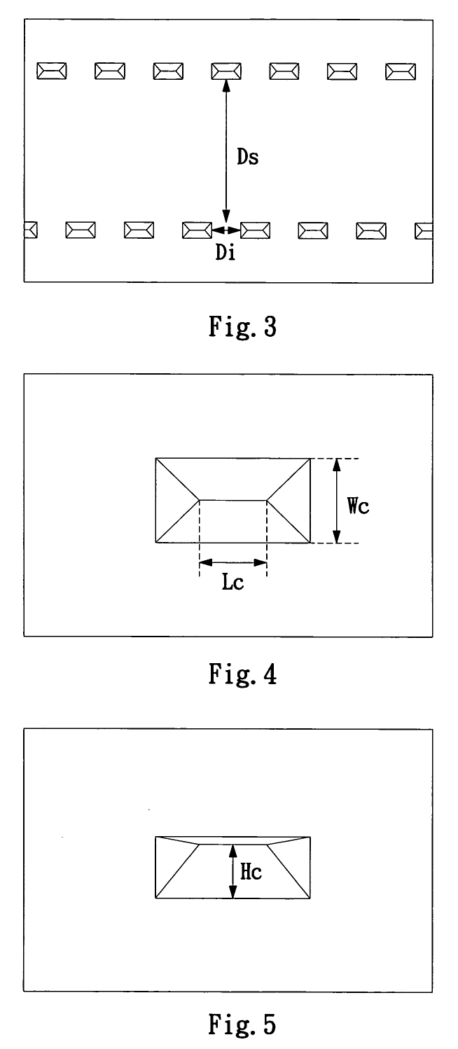

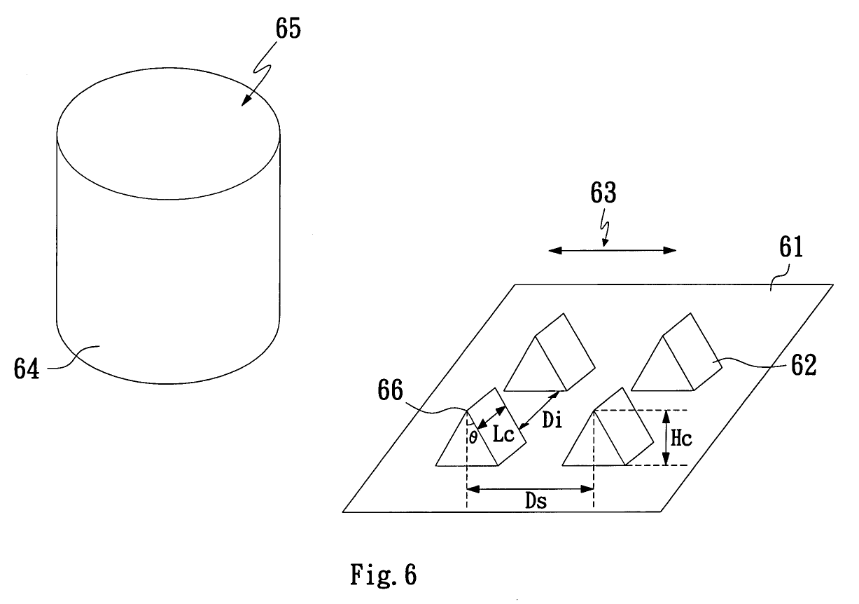

(15): Measuring the morphology and geometrical shape of the cutters with a scanning electronic microscope (SEM) and a Perthometer (Mahr Co., Germany). FIG. 3 is a top view of the microfabricated surface obtained from the above steps observed with a scanning electronic microscope; FIG. 4 is a top view of one cutter formed on the microfabricated surface of FIG. 3; and FIG. 5 is another top view of the cutter of FIG. 4. From these views, the length and width of the cutters on the processing surface may be determined. Where, Hc is the height of the cutter measured with the Perthometer. FIG. 6 is a schematic top perspective view of the microfabricated surface and the cutters obtained through the above steps. Where, reference numeral 61 indicates the processing surface, 62 indicates the cutter, 63 indicates the sliding direction of the processing surface, 64 indicates a polymeric rod material, 65 indicates a fixed load, and 64 indicates a cutting edge of the cutter 62; and the symbol θ indicates an included angle at the cutting edge of the cutter.

In the Step 2 of conducting an experiment on wear, the following steps are further included:

(21): Preparing the required material and designing the mask. That is, all the sample holder, the sample fixture, the microfabricated surface, and the polymeric rod material to be used in the experiment are dipped in a detergent and subjected to washing by supersonic oscillation for 3 minutes, washed clean using DI water, dipped in a supersonic water bath for 3 minutes, washed with DI water, dipped in 70% ethanol solution, and placed overnight to eliminate bacteria contamination. The pure water and the isopropyl alcohol solution and other solutions used in the above Step (21) must be deionized and filtered with a membrane filter having a pore size of 0.1 μm. The pure water must also be autoclaved to remove any chemical, particle, and biological contaminations;

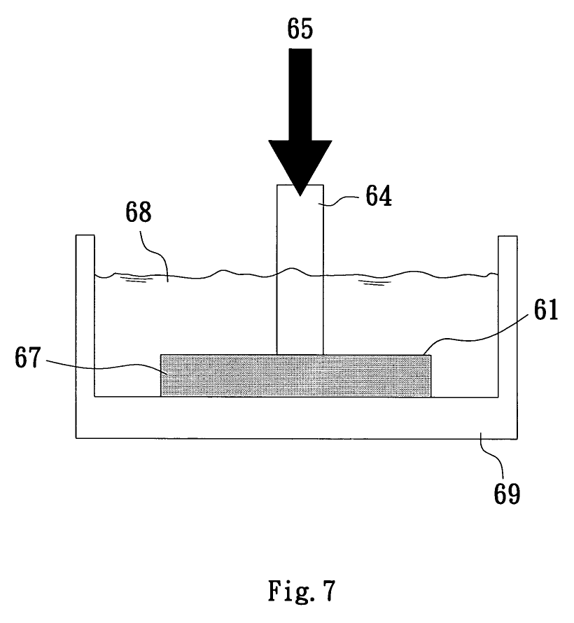

(22): Conducting a reciprocating wear test using a reciprocating wear tester shown in FIG. 7; first, adjusting the level of a wear machine and various related components to avoid two mutually abrading surfaces from bearing forces unevenly; blowing dry the UHMWPE rod material 64 with pressurized nitrogen, weighing the rod material 64 with an accuracy to the fourth decimal place, repeating the weighing three times; removing the sample holder 69, the sample fixture 67, and the microfabricated surface 61 from the 70% ethanol solution that has been placed overnight, blowing dry the same with pressurized nitrogen; positioning the UHMWPE rod material 64 in the sample fixture 67 and tightening it in place with screws; fixing the microfabricated surface 61 to the center of the sample holder 69, tightening set screws at two diagonal corners; and adjusting the stroke length Ds; Adding 4 ml of lubricating water 68 (that is, deionized water) into the sample holder 69, and wrapping the fixture 67 and the holder 69 with a clean plastic film to avoid contamination of the particles during the experiment; Placing a weight above the sample holder 69 to apply a fixed load 65 to the polymeric rod material 64 after a motor rotating speed of the wear machine has been checked and confirmed; setting the sliding speed, starting the wear tester and a time counter; monitoring the sliding speed from time to time during the test; in the case the wear procedures should exceed 24 hours, adding about 1 ml of deionized water into the sample holder 69 every 12 hours. Table 1 shows different parameters in the wear test.

TABLE 1

| | Sliding | Stroke | |
|---|---|---|---|
| Load | Speed | Length | Duration |
| 196N = 6 MPa | 57.2 mm/s | 18 mm | 24 hrs |

In the Step 3 of collecting the generated wear particles, the following steps are further included:

(31): Drawing up the wear particles generated in the wear test using a micropipette and injecting the drawn wear particles into a sterilized centrifuge test tube;

(32): Selecting a stainless steel screen having a mesh of 5 or 10 μm, mounting the same in a supersonic oscillation screener; adding a solution containing the wear particles into isopropyl alcohol, pouring the isopropyl alcohol solution into the supersonic oscillation screener to strain the large-size wear particles from the solution; and flushing the obtained wear particles with large amount of isopropyl alcohol solution to avoid particle agglomeration.

(33): Vacuum filtering the solution obtained from the above Step (32) using a polycarbonate (PC) membrane filter having a pore size of 0.1 μm in an aspirator type vacuuming system, and flushing the wall of the vacuum filtering device with isopropyl alcohol solution to avoid any wear particles from remaining on the filter;

(34): Baking out the membrane filter having the wear particles collected thereon, plating a layer of gold film on the membrane filter, observing the morphology of the wear particles with a scanning electronic microscope (SEM), and analyzing a size distribution of the wear particles with Scion image processing software to obtain a size statistics; and

(35): irradiating and sterilizing the collected wear particles with γ-ray, so that the wear particles may be used in the clinical test.

What is claimed is:

1. A method for generating polymeric wear particles, comprising the steps of:
(1) Producing a microfabricated surface;
(2) Conducting an experiment on wear; and
(3) Collecting wear particles generated in the experiment conducted in the Step (2).

2. The method for generating polymeric wear particles as claimed in claim 1, wherein the Step (1) further includes the steps of:
(11) Preparing a material and designing a mask;
(12) Forming a protective masking layer on the material by way of oxidation-diffusion;
(13) Carrying out a photolithographing process;
(14) Performing an etching process; and
(15) Measuring cutters formed on the material surface in the Step (14).

3. The method for generating polymeric wear particles as claimed in claim 2, wherein the Step (11) further includes the steps of:
(111) Selecting a suitable material as a substrate;
(112) Cleaning the substrate;
(113) Dipping the substrate in a mixture of acetone and hexane in a ratio of 1:1;
(114) Subjecting the substrate to supersonic oscillation for 10 minutes;
(115) Dipping the substrate in a deionized water for 5 minutes;
(116) Blowing dry the substrate with nitrogen; and
(117) Plotting a mask pattern.

4. The method for generating polymeric wear particles as claimed in claim 2, wherein the Step (12) further includes the steps of:
(121) Sending a silicon wafer serving as the substrate into a high-temperature reaction furnace; and
(122) Forming a silicon dioxide layer on a silicon surface of the silicon wafer through reaction of oxygen with silicon under the high temperature.

5. The method for generating polymeric wear particles as claimed in claim 2, wherein, in the Step (13), the mask pattern, which is a two-dimensional (2D) pattern, plotted in the Step (117) is transferred onto the substrate employing the photolithography.

6. The method for generating polymeric wear particles as claimed in claim 2, wherein, in the Step (14), a three-dimensional (3D) picture is produced on the substrate by etching technique.

7. The method for generating polymeric wear particles as claimed in claim 2, wherein, in the Step (15), a scanning electronic microscope (SEM) and a Perthometer are used to measure a morphology and geometrical shape of the cutters formed on the substrate.

8. The method for generating polymeric wear particles as claimed in claim 3, wherein the material for the substrate is selected from the group consisting of silicon wafer, stainless steel, high speed steel, and precision ceramics.

9. The method for generating polymeric wear particles as claimed in claim 3, wherein, in the Step (117), suitable software is used to plot the mask pattern having a feature size of 5 μm; and triangular or square mask patterns having different lengths of 60, 20, 10, and 5 μm may be separately designed.

10. The method for generating polymeric wear particles as claimed in claim 1, wherein the Step (2) further includes the steps of:
(21) Preparing required materials and designing a mask pattern; and
(22) Conducting a reciprocating wear test.

11. The method for generating polymeric wear particles as claimed in claim 10, wherein the Step (21) further includes the steps of:
(211) Dipping a sample holder, a sample fixture, a microfabricated surface produced in the Step (1), and a polymeric rod material to be used in the experiment in a detergent;
(212) Washing the items of the Step (211) by supersonic oscillation for 3 minutes;
(213) Flushing the items of the Step (212) with deionized water to clean them;
(214) Dipping the items of the Step (213) in a supersonic water bath for 3 minutes;
(215) Washing the items of the Step (214) with deionized water; and
(216) Dipping the items of the Step (215) in a 70% ethanol solution overnight to eliminate bacteria contamination.

12. The method for generating polymeric wear particles as claimed in claim 10, wherein the Step (22) further includes the steps of:
(221) Adjusting the level of a wear machine and other related components to avoid two mutually abrading surfaces from bearing forces unevenly;
(222) Blowing dry the ultra high molecular weight polyethylene (UHMWPE) rod material with pressurized nitrogen, weighing the rod material in an accuracy to at least the fourth decimal place, and repeating the weighing at least three times;
(223) Removing the sample holder, the sample fixture, and the microfabricated surface from the 70% ethanol solution that has been placed overnight, and blowing dry the items with pressurized nitrogen;
(224) Putting the UHMWPE rod material in the sample fixture, and tightening the same in place with screws; fixing the microfabricated surface to a center of the sample holder and tightening set screws at two diagonal corners; and adjusting a stroke length;
(225) Adding 4 ml of deionized water into the sample holder, wrapping the sample fixture and the sample holder with a clean plastic film to avoid contamination of particles during the experiment;
(226) Putting a weight above the sample holder to apply a fixed load to the polymeric rod material after a motor rotating speed of the wearing machine has been checked and confirmed; and setting a desired sliding speed;
(227) Starting a wear tester and a time counter; and
(228) Monitoring the sliding speed from time to time during the test, and, when the wear procedures should exceed 24 hours, adding 1 ml of deionized water into the sample holder every 12 hours.

13. The method for generating polymeric wear particles as claimed in claim 1, wherein the Step (3) further includes the steps of:
(31) Using a micropipette to draw up and inject wear particles generated in the wear test of Step (2) into a sterilized centrifuge test tube;
(32) Selecting a stainless steel screen having a mesh size of 5 or 10 μm, mounting the screen on a supersonic oscillating screener, adding a solution containing the wear particles into isopropyl alcohol, pouring the isopropyl alcohol solution into the supersonic oscillating screener to strain large-size wear particles from the solution, and flushing the obtained wear particles with large amount of isopropyl alcohol solution to avoid particle agglomeration;
(33) Vacuum filtering the solution obtained from the step (32) using a polycarbonate (PC) membrane filter having a pore size of 0.1 μm in an aspirator type vacuuming system to collect the wear particles; and flushing a wall of the vacuum filtering device with isopropyl alcohol solution to avoid attachment of any wear particles on the filter;
(34) Baking out the membrane filter having the wear particles collected thereon and then plating a layer of gold film on the membrane filter; observing a morphology of the wear particles with a scanning electronic microscope (SEM); and analyzing a size distribution of the wear particles with Scion image processing software to obtain a size statistics; and
(35) Irradiating and sterilizing the collected wear particles with γ-ray, so that the wear particles may be used in a clinical test.
